(19) United States
(12) Patent Application Publication
(10) Pub. No.: US 2025/0050086 A1
(43) Pub. Date: Feb. 13, 2025

(54) DEVICE AND METHOD FOR AUTOMATED APPLICATION OF TEMPORARY TATTOOS

(71) Applicant: Entertainment Tatts, LLC, Myrtle Beach, SC (US)

(72) Inventors: Chris MARTIN, Myrtle Beach, SC (US); Jeff DRESSING, Myrtle Beach, SC (US)

(21) Appl. No.: 18/801,081

(22) Filed: Aug. 12, 2024

Related U.S. Application Data
(60) Provisional application No. 63/532,369, filed on Aug. 12, 2023.

(51) Int. Cl. A61M 37/00

(52) U.S. Cl. CPC A61M 37/0084; A61M 2205/505; A61M 2205/6072

(57) ABSTRACT

An automated tattoo kiosk allows a tattoo to be selected and put on a person's body part. The kiosk facilitates an operator or customer in selecting and purchasing one of a variety of different digitally stored tattoos at an interactive touchscreen located at a first position of the kiosk, and issues information identifying the purchased tattoo. Then, at a second position of the kiosk, the customer provides the information identifying the purchased tattoo and a robotic arm with a spray assembly sprays the purchased tattoo on the person's skin. A computer vision system ensures that the spraying assembly is properly located near the person's body part and then sprays the selected tattoo on their skin.

This application claims priority to U.S. Provisional Patent Application No. 63/532,369 filed on Aug. 12, 2023, the specification and drawings of which are hereby incorporated by reference in their entirety.

### FIELD OF THE INVENTION

The present disclosure lies in the general field of body art. More specifically, it relates to apparatus and methods for automatically applying a temporary tattoo to a person's skin.

### BACKGROUND

Various prior attempts have been made to expedite the process of creating body art where ink or similar substance is injected into or onto parts of the skin. In PCT Patent Publication No. WO 2015/193513 by Johan Da Silveira, it was suggested that an automated tattoo needle can be used to apply a requisite combination of puncture and ink at a design marked or printed onto a person's skin to make a tattoo. In United States Patent Application Publication No. 2022/0313970A1 by Yan Azdoud et al, a “stencil” is applied to the skin to guide a robotically controlled needle to its appropriate positions. The “stencil” is comprised of a plurality of dot positions. The sum of all of the dot positions creates the desired image of the entire tattoo. The robotic needle senses the location of each dot and applies the required skin puncture/ink injection combination, and then repeats that process with all dot positions in the tattoo design. In PCT Patent Publication No. WO 2020/178818A1 by Orr Karl Fabian, images of the body part's skin are captured, and then an image of the tattoo design is overlayed onto that body part virtually to confirm the desired appearance and location of the design. A robotic needle applies the required skin puncture/ink injection combination, following the pattern of the virtual image remembered by the controller. These and other similar systems and methods involve injections of ink into the skin, and are intended to expedite the creation of permanent tattoos. But they have largely proven unsuccessful for technical reasons and the resistance by customers to submit to a robotic needle puncturing their skin.

It is also known in the field of face painting and body art for an artist to hover a spray gun over a person's skin and move it while pulling a trigger mechanism to release the spray (under pressure with an air compressor) to apply ink in a selective fashion to a person's skin (this is sometimes referred to as “air-brush”). Such a method requires human expertise and can be somewhat time consuming. In U.S. Pat. No. 10,621,812 by Patrick McNenny, an automated system was described in which a print head prints a digitally stored tattoo onto the skin of a body part inserted into an aperture of a kiosk or a mechanical arm having a print head is moved to print on a body part. This prior art can utilize ink that does not require a needle to puncture the skin, but it also has disadvantages that make it undesirable, particularly to children and families.

### BRIEF SUMMARY

An object of the preferred embodiments is to provide an automated system to provide temporary tattoos in a quick and aesthetically pleasing manner. More specifically the preferred embodiments are a self-service, interactive kiosk that applies a temporary tattoo to a selected part of a person's skin, using an automated air-brush spraying mechanism. Temporary ink that wears off and can be removed with a cleanser is sprayed onto a part of the skin designated by the person.

### BRIEF DESCRIPTION OF THE DRAWINGS

FIG. 1 is a front view of a kiosk according to the preferred embodiment having a robotic arm within the enclosure.

FIG. 2 shows a preferred embodiment of the kiosk having two interactive touchscreens on the sides of the kiosk.

FIG. 3 is a front view of the preferred embodiment of the kiosk having a multi-axis robotic arm mounted on a platform in the center of the enclosure.

FIG. 4 shows the airbrush tattoo sprayer assembly used to apply ink for the temporary tattoos in the preferred embodiment.

FIG. 5 shows the computer vision system of the preferred embodiment.

### DETAILED DESCRIPTION OF THE PREFERRED EMBODIMENTS

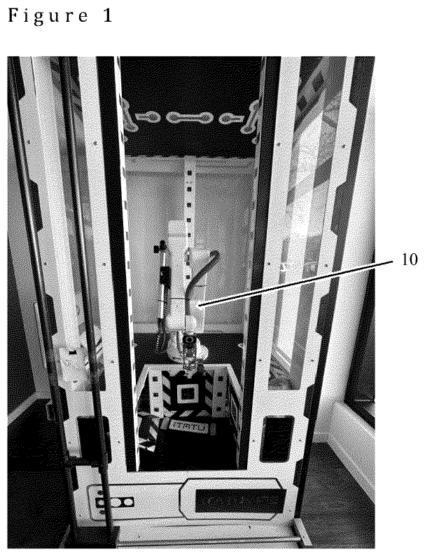

The preferred embodiments of the invention are an automated, interactive, kiosk applying a temporary tattoo. In a preferred embodiment shown in FIG. 1, the kiosk is a self-service kiosk that applies a temporary tattoo to part of a person's skin using an automated air-brush spraying mechanism.

The preferred embodiments use an outline of the shape which defines the edges of the desired tattoo image. That outline is an adhesive film that has a solid rectangular border, with the shape of the tattoo image laser-cut and removed, leaving a “gap” that exposes the skin when the outline is applied to the skin. That outline has a “home” marker printed on the edge of the rectangle which is not cut and removed. The robotic airbrush sprayer senses/detects the home location on the outline, and uses that relative reference coordinate to spray the appropriate ink(s) in a sweeping motion across the gap in the outline, so the ink adheres to the exposed skin

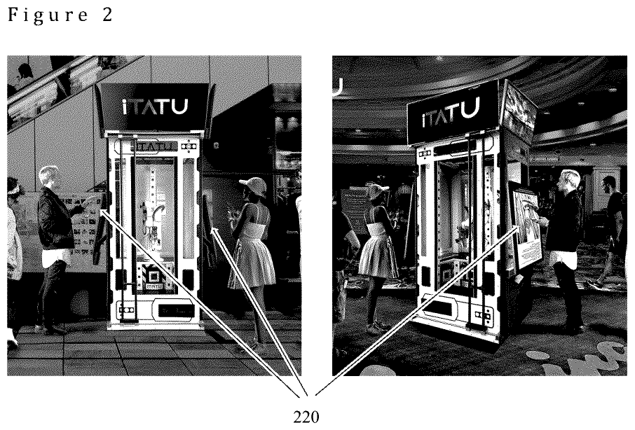

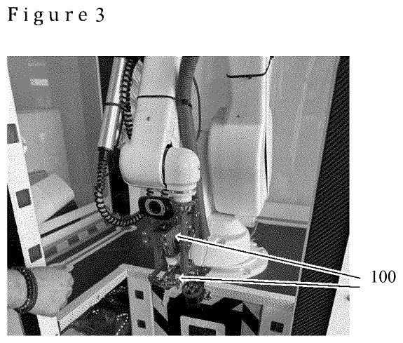

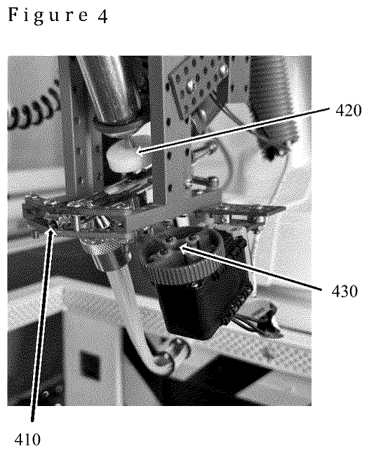

In the preferred embodiments, the kiosk is supported by wooden base and frame in a rectangular shape to define a largely open air enclosure, though other shapes and building materials may be used. Attached to the wood frame are plastic frames which hold plexiglass in place to provide visibility to the robotic arm inside the enclosure. See the top view of the enclosure in FIGS. 4-6 in the provisional application. In the front of the preferred embodiment, there is a sliding 2-railed grip, extending from the base to the top, that slides across the front of the device, and which is preferably used to help position customers' body parts during the spraying process. See FIGS. 2 and 3 in the provisional application. The base of the rail mechanism supports two vertical rods that extend to the top of the enclosure, where the rods are supported by another identical base. Both bases slide horizontally on a single track (see 7a of FIG. 7 in the provisional application), which travels the width of the kiosk base.

In the preferred embodiments, the kiosk has an interactive computer touchscreen 220 on one side on the enclosure, and preferably one on the other side of the enclosure as well as shown in FIG. 2. Optionally, there may be two order touchscreens on each side of the kiosk (for a total of four), or other order touchscreens which are located nearby and physically separate from the kiosk to support respective multiple queues of customers. A human-machine interface (HMI) is supported by each one of the touchscreens and permits an operator/assistant, or the consumer where the kiosk is a self-service kiosk, to select the size/type/color of the temporary tattoo to be applied. Multiple configurations of the HMI are possible and there may be multiple themes selectable according to the environment. For example, if the kiosk is to be deployed at a football game, then the screen may have a football theme.

In addition to selecting their tattoo, customers also preferably complete payment at the touchscreen 220 via a suitable web-based payment application. In one embodiment, the kiosk may provide the customer with a unique order number, QR code, barcode, or other simple identifying information. It can be provided by printing or be transmitted to the customer's cell phone. The customer will then take that identifying information to the front of the kiosk to be scanned by a scanner or otherwise input, which will initiate the physical tattoo application process. Therefore, in this embodiment, the automated tattoo process is separated into two different interactions and queues—a first one with touchscreen(s) for selecting and payment of the tattoo and a second one with a QR code reader (or other input device) for the physical tattoo application process. The preferred tattoo application process disclosed herein takes less than one minute. The inventors have determined that customers typically take considerably longer than one minute to select their tattoo design. Thus, the separation of the two processes increases the kiosk's productivity and profitability which is primarily proportional to the number of tattoos sold and applied during an hour.

FIG. 3 is a front view of the preferred embodiments of the kiosk having a multi-axis robotic arm mounted on a platform in the center of the enclosure. The end effector attached at the end of the robotic arm is an airbrush sprayer device and accompanying ink containers 100. The arm has control wires connected to a robotic controller, which is located at the base of the platform (see FIG. 11 in the provisional application) or under the platform. Ink containers (cylindrical cartridges) are mounted on the side of the robotic arm and there are conduits for the compressed air tube which propels the ink via the sprayer and the control wires that operate the sprayer actuators. (see FIGS. 8 and 9 in the provisional application). Although most of the temporary tattoos are relatively small, the robotic arm preferably carries out a calibration routine in which it moves in all directions. For this reason, the enclosure provides a protected envelope of clearance space around the robotic arm and attached sprayer.

FIG. 4 shows the airbrush tattoo sprayer assembly used to apply ink for the temporary tattoos in the preferred embodiments. The sprayer is attached to the robotic arm, which is equipped with an actuator to activate the trigger on the sprayer. While the arm sweeps back and forth, the control system activates the trigger to release the spray (under pressure with an air compressor located under the robotic arm platform) to apply the ink to the body part. Preferably, the kiosk also has an actuator mechanism to change the ink (selecting from 8 or more different colors, as well as cleaning solutions) which is stored in the cylindrical cartridges attached to the sides of the robotic arm. The ink is emitted at sprayer nozzle 410. Actuator arm 420 depressed the sprayer valve to release the air which sprays the ink. Actuated gear 430 rotates to position the desired ink color tube into the correct position to be sprayed by the compressed air. There may also be a consolidation harness of ink tubes coming from the ink containers on the side of the robotic arm. (see FIG. 14 in the provisional application). These are preferably joined in a harness that can rotate via actuated gear 430.

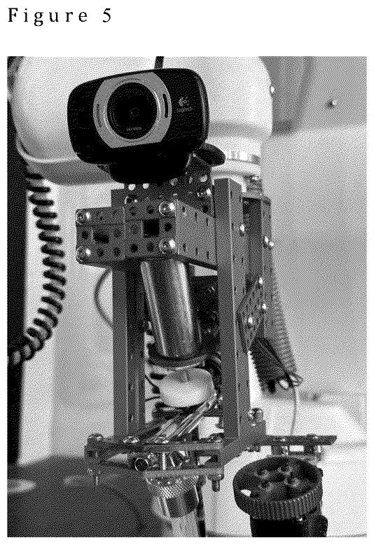

The preferred embodiments have a computer vision system via a camera mounted on the end of the robotic arm and attached to the top of the sprayer assembly as shown in FIG. 5. The camera provides video input to the vision system which provides a visual confirmation feedback control to ensure the customer's body part is in the correct location. Preferably, there is visual display feedback so that a customer can more easily and/or accurately align their body part in the correct alignment/direction. The computer vision system may optionally be assisted by sensors to ensure customer is in the correct location and position before starting application of the tattoo spray. These sensors could comprise, for example, weight-activated sensor mats where customer stands at the front of the kiosk, and grip/touch sensors where customer holds on to the kiosk exterior. Preferably, spraying of the tattoo commences when the customer confirms that the location is correct.

In the preferred embodiments, the components of the control system are preferably located under the robotic arm platform in the base of the kiosk. The components include a robotic arm controller, a PLC-type device to control actuators, and a computer device. A software application in the computer device integrates the user interface and the controllers.

In another embodiment, the tattoo application process uses an outline through which the ink is sprayed to create the intended design on the skin. The pre-made outlines are made available beside the machine. The kiosk may have components to create and dispense the outlines to customers, who will then apply the outline to their skin. In one particular example, the kiosk prints the QR code for a customer's order on the outline and the QR code is read by the vision system prior to commencing spraying.

Using the kiosk of the preferred embodiments disclosed above, a method of applying a tattoo ordered by a customer includes the following steps.

1. The customer uses the human machine interface (HMI) associated with a kiosk to choose a tattoo, defined as combination of (a) design/artwork/style (e.g., a logo, a shark, heart, arrow, etc.), (b) size, (c) color(s), and (d) body part.

2. The HMI system communicates the order info to the control system of the kiosk.

3. A video screen on the front of the kiosk displays instructions about where to stand, and how to apply the optional outline to the appropriate body part.

4. The customer applies the outline received from the kiosk to the body part.

5. The customer moves into the proper position based on instructions shown on the kiosk video screen.

6. A vision system confirms that the customer is in the correct position.

7. A control system activates the instructions to begin applying the tattoo, including the following: (a) move the robotic arm to the correct starting location based on body part and vision system confirmation, (b) the vision system looks for the QR code on the outline to confirm order ID, (c) the spray color actuator is indexed to the correct position, (d) the robotic arm moves in a sweeping pattern and the actuator pulls spray trigger at appropriate time during sweeping pattern, (e) If required, the spray color actuator is indexed to the next correct position, and (f) repeat sub-step € until the tattoo is completed.

8. The robotic arm returns to its home position and performs cleaning routine to clear nozzles.

9. When the tattoo is complete, the kiosk front video screen notifies the customer and may display instructions for removing the outline. There may also be displayed instructions for later cleaning and/or removing of the tattoo.

We hereby claim as our invention:

1. An automated tattoo kiosk, comprising:
an upright enclosure, said enclosure generally having a front and two sides and defining a space therein for the application of a tattoo to a person's skin;
at least one display touchscreen located at a first location of the enclosure, said display touchscreen having an interface through which one of a plurality of tattoo designs can be selected and purchased;
a system for providing the customer with information identifying the purchased tattoo design at the location of said at least one display touchscreen;
a second location of the enclosure at which the information identifying the purchased tattoo and the body part to receive the purchased tattoo can be presented;
a movable robotic arm within the enclosure space;
a spray assembly attached to the end of the movable robotic arm; and
a control system which, when the information identifying the purchased tattoo design and the body part are presented at the second location, moves the robotic arm to locate the spray assembly near the body part and sprays the tattoo on the customer's skin.

2. The kiosk recited in claim 1, wherein the second location contains a display for displaying instructions to the customer.

3. The kiosk recited in claim 1, wherein the information identifying the purchased tattoo design comprises a QR code and the second location of the enclosure includes a QR code scanner.

4. The kiosk recited in claim 1, wherein the information identifying the purchased tattoo design comprises a number and the second location of the enclosure allows the customer to input the number to initiate the tattoo application process.

5. The kiosk recited in claim 1, further comprising a computer vision system to determine the position of the spray assembly so as to be near the customer's body part to receive the purchased tattoo.

6. The kiosk recited in claim 5, wherein the computer vision system is assisted by sensors located in the kiosk.

7. The kiosk recited in claim 5 wherein the control system does not begin to spray the purchased tattoo design until the customer has confirmed the location of their body part and the spray assembly.

8. The kiosk recited in claim 5, wherein the enclosure includes plexiglass and the customer can view the spray assembly and their body part during the tattoo application process.

9. The kiosk recited in claim 1, wherein the kiosk provides an outline of the purchased tattoo design, and the purchased tattoo is sprayed on the customer's skin within the outline.

10. The kiosk recited in claim 9, wherein the information identifying the purchased tattoo design is printed on the outline.

11. A method of applying a tattoo, comprising:
providing an interface on a touchscreen at a first location of a kiosk for a customer to select one of a plurality of tattoo designs;
providing information identifying the selected tattoo design at the first location and an outline of the selected design;
when the information identifying the selected tattoo design is presented at a second location of the kiosk, instructing the customer how to apply the outline and position their body part;
confirming the position of the customer's body part;
applying the selected tattoo design to the body part in the outline by a spray assembly moved by a robotic arm;
returning the robotic arm and spray assembly to its home position; and
displaying instructions to the customer at the second location of the kiosk.

12. The method recited in claim 11, wherein the position of the customer's body part is confirmed by a computer vision system.

13. The method recited in claim 12, wherein the computer vision system is assisted by sensors located in the kiosk.

14. The kiosk recited in claim 12, wherein the selected tattoo design is not sprayed until the customer has confirmed the location of their body part and the spray assembly.
